(19) United States
(12) Patent Application Publication
(10) Pub. No.: US 2014/0093585 A1
(43) Pub. Date: Apr. 3, 2014

(54) PARP INHIBITORS FOR THE TREATMENT OF CIPN

(71) Applicant: AbbVie Inc., North Chicago, IL (US)

(72) Inventors: Vincent L. Giranda, Gurnee, IL (US); Alexander R. Shoemaker, Green Oaks, IL (US); Kaitlin E. Browman, Deerfield, IL (US); Shailen K. Joshi, Grayslake, IL (US); Jill-Desiree Brederson, Evanston, IL (US)

(21) Appl. No.: 14/098,128

(22) Filed: Dec. 5, 2013

Related U.S. Application Data
(63) Continuation of application No. 13/646,856, filed on Oct. 8, 2012, now abandoned.
Application No. 13/646,856 is a continuation-in-part of application No. 13/442,294, filed on Apr. 9, 2012, now abandoned.
(60) Provisional application No. 61/473,970, filed on Apr. 11, 2011.
(60) Provisional application No. 61/476,616, filed on Apr. 18, 2011.

(51) Int. Cl. C07D 403/04; A61K 33/24; A61K 31/7068; A61K 31/4184; A61K 31/555; A61K 31/475; A61K 31/337

(52) U.S. Cl. CPC C07D 403/04; A61K 31/475; A61K 33/24; A61K 31/337; A61K 31/4184; A61K 31/555; A61K 31/7068
USPC 424/649; 514/394; 514/283; 514/49

(57) ABSTRACT

The present invention relates to the method of treating chemotherapy-induced neuropathy in a subject in need thereof with the use of a poly(ADP-ribose)polymerase (PARP) inhibitor.

### CROSS-REFERENCES TO RELATED APPLICATIONS

This application is a continuation-in-part of U.S. application Ser. No. 13/442,294, filed on Apr. 9, 2012, which claims priority to provisional application Ser. No. 61/473,970, filed on Apr. 11, 2011, and provisional application Ser. No. 61/476,616, filed on Apr. 18, 2011, the contents of each of which are herein incorporated by reference.

### FIELD OF THE INVENTION

This invention pertains to the use of poly(ADP-ribose)polymerase (PARP) inhibitors for the treatment and/or prevention of chemotherapy-induced peripheral neuropathy (CIPN).

### BACKGROUND OF THE INVENTION

Chemotherapy-induced peripheral neuropathy (CIPN) is a disabling side effect of many chemotherapeutic agents. Symptoms are sensory, or a combination of sensory and motor, and include numbness, tingling, pins and needles, burning, decreased or altered sensation, painful increase in sensitivity in the hands and feet, and/or motor weakness. (Hausheer F. H. et al., Semin Oncol 2006 33:15-49). CIPN can be acute or persistent and result in compromised daily functioning and quality of life. (Postma T. J. et al., European Journal of Cancer 2005 41:1135-1139).

CIPN is associated with chemotherapeutic agents such as platinum-based agents, vinca-alkaloids, and taxanes, and is often the dose-limiting side effect of these agents. (Hausheer F. H. et al., Semin Oncol 2006 33:15-49). The incidence of CIPN is highly variable and can depend on many factors, including dose, cumulative dose, duration of the chemotherapy, combination therapy with other chemotherapeutic agents, as well as age and the presence of a high-risk pre-existing condition, e.g., diabetes. (Wolf S., et al., European Journal of Cancer 2008 44:1507-1515; Nurgalieva Z., et al., American Journal of Therapeutics 2010 17:148-158; Hausheer F. H. et al., Semin Oncol 2006 33:15-49). For example, the incidence of CIPN reported on the carboplatin label ranges from 6% to 42%, and the incidence of CIPN reported on the paclitaxel label ranges from 42% to 79%.

There is a lack of effective strategies for preventing CIPN or treating established CIPN. Currently, the standard therapy consists of reducing exposure to the toxic substance, followed by symptomatic and supportive therapy, e.g., tricyclic antidepressants, anticonvulsants, opioids or NSAIDs for pain and other symptoms. (Kaley, T. J. et al., British Journal of Haematology 2009 145:3-13). Thus, development of CIPN can result in dose modifications and interruptions, delays, or even complete cessation of the chemotherapy, adversely affecting treatment of the malignancy and patient outcome.

There remains a need for effective methods for the treatment, prophylactic treatment, and/or mitigation of CIPN and its symptoms. In addition, there remains a need for effective methods for the treatment, prophylactic treatment, and/or mitigation of CIPN and its symptoms without interference with the anti-tumor activity of the chemotherapy.

### BRIEF SUMMARY OF THE INVENTION

The present invention relates to a method for the treatment of chemotherapy-induced peripheral neuropathy in a subject, comprising administering to the subject an effective amount of a compound of formula (I):

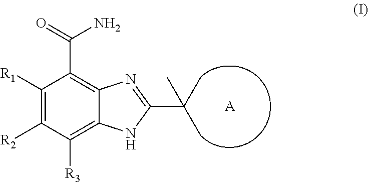

or a pharmaceutically acceptable salt or solvate thereof, wherein

R₁, R₂, and R₃ are independently selected from the group consisting of hydrogen, alkenyl, alkoxy, alkoxycarbonyl, alkyl, alkynyl, cyano, haloalkoxy, haloalkyl, halogen, hydroxy, hydroxyalkyl, nitro, NR_{A}R_{B}, and (NR_{A}R_{B})carbonyl;

A is a nonaromatic 4, 5, 6, 7, or 8-membered ring that contains 1 or 2 nitrogen atoms and, optionally, one sulfur or oxygen atom, wherein the nonaromatic ring is optionally substituted with 1, 2, or 3 substituents selected from the group consisting of alkenyl, alkoxy, alkoxyalkyl, alkoxycarbonyl, alkoxycarbonylalkyl, alkyl, alkynyl, aryl, arylalkyl, cycloalkyl, cycloalkylalkyl, cyano, haloalkoxy, haloalkyl, halogen, heterocycle, heterocyclealkyl, heteroaryl, heteroarylalkyl, hydroxy, hydroxyalkyl, nitro, NR_{C}R_{D}, (NR_{C}R_{D})alkyl, (NR_{C}R_{D})carbonyl, (NR_{C}R_{D})carbonylalkyl, (NR_{C}R_{D})sulfonyl, and oxo; and R_{A}, R_{B}, R_{C}, and R_{D} are independently selected from the group consisting of hydrogen, alkyl, and alkycarbonyl.

The present invention relates to a method for prophylactic treatment of chemotherapy-induced peripheral neuropathy in a subject, comprising administering to the subject an effective amount of a compound of formula (I).

The present invention relates to a method for mitigating neurotoxic effects of a chemotherapeutic agent, comprising administering to a subject an effective amount of a compound of formula (I).

The present invention relates to a method for treating chemotherapy-induced neuropathic pain in a subject, comprising administering to the subject an effective amount of a compound of formula (I).

The present invention also relates to a method for the treatment of chemotherapy-induced peripheral neuropathy in a subject, comprising administering to the subject an effective amount of a compound of formula (II):

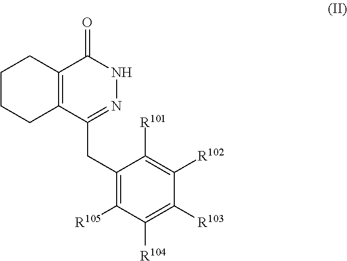

or a pharmaceutically acceptable salt or solvate thereof, wherein

### R₁₀₁, R₁₀₄, and R₁₀₅ are H;

### R₁₀₃ is F;

R₁₀₂ is selected from pyrrolidinyl, oxazolyl, imidazolidinyl, isothiazolidinyl, piperidinyl, piperazinyl and azepanyl, wherein R₁₀₂ is substituted with one or two (O) substituents.

The present invention relates to a method for prophylactic treatment of chemotherapy-induced peripheral neuropathy in a subject, comprising administering to the subject an effective amount of a compound of formula (II).

The present invention relates to a method for mitigating neurotoxic effects of a chemotherapeutic agent, comprising administering to a subject an effective amount of a compound of formula (II).

The present invention relates to a method for treating chemotherapy-induced neuropathic pain in a subject, comprising administering to the subject an effective amount of a compound of formula (II).

### BRIEF DESCRIPTION OF THE DRAWINGS

FIG. 1 shows the prophylactic effect of 2-[(2R)-2-methylpyrrolidin-2-yl]-1H-benzimidazole-4-carboxamide (Compound A) on vincristine-mediated neuropathy manifested by the prevention of vincristine-mediated pain.

FIG. 2 shows the prophylactic effect of 6-fluoro-2-(2-methylpyrrolidin-2-yl)-1H-benzimidazole-4-carboxamide (Compound B) on vincristine-mediated neuropathy manifested by the prevention of vincristine-mediated pain.

FIG. 3 shows the prophylactic effect of 2-[(2S)-2-methylpyrrolidin-2-yl]-1H-benzimidazole-4-carboxamide (Compound C) on oxaliplatin-mediated neuropathy manifested by the attenuation of oxaliplatin-mediated mechanical allodynia.

FIG. 4 shows the prophylactic effect of 2-[(2S)-2-methylpyrrolidin-2-yl]-1H-benzimidazole-4-carboxamide (Compound C) on oxaliplatin-mediated neuropathy manifested by the attenuation of oxaliplatin-mediated cold allodynia.

FIG. 5 shows the prophylactic effect of 2-[(2S)-2-methylpyrrolidin-2-yl]-1H-benzimidazole-4-carboxamide (Compound C) on cisplatin-mediated neuropathy manifested by the attenuation of cisplatin-mediated mechanical allodynia.

FIG. 6 shows the prophylactic effect of 2-[(2S)-2-methylpyrrolidin-2-yl]-1H-benzimidazole-4-carboxamide (Compound C) on cisplatin-mediated neuropathy manifested by the attenuation of cisplatin-mediated heat hyperalgesia.

FIG. 7 shows the prophylactic effect of 2-[(2R)-2-methylpyrrolidin-2-yl]-1H-benzimidazole-4-carboxamide (Compound A) on vincristine-mediated neuropathy manifested by the prevention of vincristine-mediated pain.

FIG. 8 shows the prevention of vincristine-mediated increase in PAR levels in skin by treatment with 2-[(2R)-2-methylpyrrolidin-2-yl]-1H-benzimidazole-4-carboxamide (Compound A).

FIG. 9 shows the prophylactic effect of 2-[(2S)-2-methylpyrrolidin-2-yl]-1H-benzimidazole-4-carboxamide (Compound C) on cisplatin-induced decrease in amplitude in digital nerve SNAP recording.

FIG. 10 shows the prophylactic effect of 1-(2-fluoro-5-((4-oxo-3,4,5,6,7,8-hexahydrophthalazin-1-yl)methyl)phenyl)pyrrolidine-2,5-dione (Compound D) on vincristine-mediated neuropathy manifested by the prevention of vincristine-mediated pain.

### DETAILED DESCRIPTION OF THE INVENTION

### Definitions

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. In this application, the use of “or” means “and/or” unless stated otherwise. Furthermore, the use of the term “including”, as well as other forms, such as “includes” and “included”, is not limiting. With reference to the use of the words “comprise” or “comprises” or “comprising” in this patent application (including the claims), Applicants note that unless the context requires otherwise, those words are used on the basis and clear understanding that they are to be interpreted inclusively, rather than exclusively, and that Applicants intend each of those words to be so interpreted in construing this patent application, including the claims below. For a variable that occurs more than one time in any substituent or in the compound of the invention or any other formulae herein, its definition on each occurrence is independent of its definition at every other occurrence.

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkyl” as used herein, means at least one alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkoxycarbonyl” as used herein, means an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkoxycarbonylalkyl” as used herein, means an alkoxycarbonyl group, as defined herein, appended to the parent molecular moiety through an alkyl group, as defined herein.

The term “alkyl” as used herein, means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylcarbonyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonyloxy” as used herein, means an alkylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy.

The term “alkylthio” as used herein, means an alkyl group, as defined herein, appended to the parent molecular moiety through a sulfur atom. Representative examples of alkylthio include, but are not limited, methylthio, ethylthio, tert-butylthio, and hexylthio.

The term “alkylthioalkyl” as used herein, means an alkylthio group, as defined herein, appended to the parent molecular moiety through an alkyl group, as defined herein. Representative examples of alkylthioalkyl include, but are not limited, methylthiomethyl and 2-(ethylthio)ethyl.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl,” as used herein, means a phenyl group or a naphthyl group.

The aryl groups of the present invention can be optionally substituted with one, two, three, four, or five substituents independently selected from the group consisting of alkenyl, alkoxy, alkoxyalkyl, alkoxycarbonyl, alkyl, alkylcarbonyl, alkylcarbonyloxy, alkylthio, alkylthioalkyl, alkynyl, carboxy, cyano, formyl, haloalkoxy, haloalkyl, halogen, hydroxy, hydroxyalkyl, mercapto, nitro, —NR_{E}R_{F}, and (NR_{E}R_{F})carbonyl.

The term “arylalkyl” as used herein, means an aryl group, as defined herein, appended to the parent molecular moiety through an alkyl group, as defined herein. Representative examples of arylalkyl include, but are not limited to, benzyl, 2-phenylethyl, 3-phenylpropyl, 1-methyl-3-phenylpropyl, and 2-naphth-2-ylethyl.

The term “carbonyl” as used herein, means a —C(O)— group.

The term “carboxy” as used herein, means a —CO₂H group.

The term “cyano” as used herein, means a —CN group.

The term “cycloalkyl” as used herein, means a saturated cyclic hydrocarbon group containing from 3 to 8 carbons, examples of cycloalkyl include cyclopropyl, cyclobutyl, cyclopentyl, cyclohexyl, cycloheptyl, and cyclooctyl.

The cycloalkyl groups of the present invention are optionally substituted with 1, 2, 3, or 4 substituents selected from alkenyl, alkoxy, alkoxyalkyl, alkoxycarbonyl, alkyl, alkylcarbonyl, alkylcarbonyloxy, alkylthio, alkylthioalkyl, alkynyl, carboxy, cyano, formyl, haloalkoxy, haloalkyl, halogen, hydroxy, hydroxyalkyl, mercapto, oxo, —NR_{E}R_{F}, and (NR_{E}R_{F})carbonyl.

The term “cycloalkylalkyl” as used herein, means a cycloalkyl group, as defined herein, appended to the parent molecular moiety through an alkyl group, as defined herein. Representative examples of cycloalkylalkyl include, but are not limited to, cyclopropylmethyl, 2-cyclobutylethyl, cyclopentylmethyl, cyclohexylmethyl, and 4-cycloheptylbutyl.

The term “formyl” as used herein, means a —C(O)H group.

The term “halo” or “halogen” as used herein, means —Cl, —Br, —I or —F.

The term “haloalkoxy” as used herein, means at least one halogen, as defined herein, appended to the parent molecular moiety through an alkoxy group, as defined herein.

Representative examples of haloalkoxy include, but are not limited to, chloromethoxy, 2-fluoroethoxy, trifluoromethoxy, and pentafluoroethoxy.

The term “haloalkyl” as used herein, means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heteroaryl,” as used herein, means a monocyclic heteroaryl ring or a bicyclic heteroaryl ring. The monocyclic heteroaryl ring is a 5 or 6 membered ring. The 5 membered ring has two double bonds and contains one, two, three or four heteroatoms independently selected from the group consisting of N, O, and S. The 6 membered ring has three double bonds and contains one, two, three or four heteroatoms independently selected from the group consisting of N, O, and S. The bicyclic heteroaryl ring consists of the 5 or 6 membered heteroaryl ring fused to a phenyl group or the 5 or 6 membered heteroaryl ring is fused to another 5 or 6 membered heteroaryl ring. Nitrogen heteroatoms contained within the heteroaryl may be optionally oxidized to the N-oxide. The heteroaryl is connected to the parent molecular moiety through any carbon atom contained within the heteroaryl while maintaining proper valence. Representative examples of heteroaryl include, but are not limited to, benzothienyl, benzoxadiazolyl, cinnolinyl, furopyridinyl, furyl, imidazolyl, indazolyl, indolyl, isoxazolyl, isoquinolinyl, isothiazolyl, naphthyridinyl, oxadiazolyl, oxazolyl, pyridinyl, pyridazinyl, pyrimidinyl, pyrazinyl, pyrazolyl, pyrrolyl, pyridinium N-oxide, quinolinyl, tetrazolyl, thiadiazolyl, thiazolyl, thienopyridinyl, thienyl, triazolyl, and triazinyl.

The heteroaryl groups of the present invention are substituted with 0, 1, 2, 3, or 4 substituents independently selected from alkenyl, alkoxy, alkoxyalkyl, alkoxycarbonyl, alkyl, alkylcarbonyl, alkylcarbonyloxy, alkylthio, alkylthioalkyl, alkynyl, carboxy, cyano, formyl, haloalkoxy, haloalkyl, halogen, hydroxy, hydroxyalkyl, mercapto, nitro, —NR_{E}R_{F}, and (NR_{E}R_{F})carbonyl.

The term “heteroarylalkyl” as used herein, means a heteroaryl, as defined herein, appended to the parent molecular moiety through an alkyl group, as defined herein. Representative examples of heteroarylalkyl include, but are not limited to, pyridinymethyl.

The term “heterocycle” or “heterocyclic” as used herein, means a monocyclic or bicyclic heterocyclic ring. The monocyclic heterocyclic ring consists of a 3, 4, 5, 6, 7, or 8 membered ring containing at least one heteroatom independently selected from O, N, and S. The 3 or 4 membered ring contains 1 heteroatom selected from the group consisting of O, N and S. The 5 membered ring contains zero or one double bond and one, two or three heteroatoms selected from the group consisting of O, N and S. The 6 or 7 membered ring contains zero, one or two double bonds and one, two or three heteroatoms selected from the group consisting of O, N and S. The bicyclic heterocyclic ring consists of a monocyclic heterocyclic ring fused to a cycloalkyl group or the monocyclic heterocyclic ring fused to a phenyl group or the monocyclic heterocyclic ring fused to another monocyclic heterocyclic ring. The heterocycle is connected to the parent molecular moiety through any carbon or nitrogen atom contained within the heterocycle while maintaining proper valence. Representative examples of heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyrazolinyl, pyrazolidinyl, pyrrolinyl, pyrrolidinyl, tetrahydrofuranyl, tetrahydropyranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl.

The heterocycles of this invention are substituted with 0, 1, 2, or 3 substituents independently selected from alkenyl, alkoxy, alkoxyalkyl, alkoxycarbonyl, alkyl, alkylcarbonyl, alkylcarbonyloxy, alkylthio, alkylthioalkyl, alkynyl, carboxy, cyano, formyl, haloalkoxy, haloalkyl, halogen, hydroxy, hydroxyalkyl, mercapto, nitro, —NR_{E}R_{F}, and (NR_{E}R_{F})carbonyl.

The term “heterocyclealkyl” as used herein, means a heterocycle, as defined herein, appended to the parent molecular moiety through an alkyl group, as defined herein.

The term “hydroxy” as used herein, means an —OH group.

The term “hydroxyalkyl” as used herein, means at least one hydroxy group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “mercapto” as used herein, means a —SH group.

The term “nitro” as used herein, means a —NO₂ group.

The term “nonaromatic” as used herein, means that a 4 membered nonaromatic ring contains zero double bonds, a 5 membered nonaromatic ring contains zero or one double bond, a 6, 7, or 8 membered nonaromatic ring contains zero, one, or two double bonds.

The term “NR_{A}R_{B}” as used herein, means two groups, R_{A} and R_{B}, which are appended to the parent molecular moiety through a nitrogen atom. R_{A} and R_{B} are each independently hydrogen, alkyl, and alkylcarbonyl. Representative examples of NR_{A}R_{B} include, but are not limited to, amino, methylamino, acetylamino, and acetylmethylamino.

The term “(NR_{A}R_{B})carbonyl” as used herein, means a NR_{A}R_{B} group, as defined herein, appended to the parent molecular moiety through a carbonyl group, as defined herein. Representative examples of (NR_{A}R_{B})carbonyl include, but are not limited to, aminocarbonyl, (methylamino)carbonyl, (dimethylamino)carbonyl, and (ethylmethylamino)carbonyl.

The term “NR_{C}R_{D}” as used herein, means two groups, R_{C} and R_{D}, which are appended to the parent molecular moiety through a nitrogen atom. R_{C} and R_{D} are each independently hydrogen, alkyl, and alkylcarbonyl. Representative examples of NR_{C}R_{D} include, but are not limited to, amino, methylamino, acetylamino, and acetylmethylamino.

The term “(NR_{C}R_{D})carbonyl” as used herein, means a NR_{C}R_{D} group, as defined herein, appended to the parent molecular moiety through a carbonyl group, as defined herein. Representative examples of (NR_{C}R_{D})carbonyl include, but are not limited to, aminocarbonyl, (methylamino)carbonyl, (dimethylamino)carbonyl, and (ethylmethylamino)carbonyl.

The term “(NR_{C}R_{D})carbonylalkyl” as used herein, means a (NR_{C}R_{D})carbonyl group, as defined herein, appended to the parent molecular moiety through an alkyl group, as defined herein.

The term “(NR_{C}R_{D})sulfonyl” as used herein, means a NR_{C}R_{D} group, as defined herein, appended to the parent molecular moiety through a sulfonyl group, as defined herein. Representative examples of (NR_{C}R_{D})sulfonyl include, but are not limited to, aminosulfonyl, (methylamino)sulfonyl, (dimethylamino)sulfonyl, and (ethylmethylamino)sulfonyl.

The term “NR_{E}R_{F}” as used herein, means two groups, R_{E} and R_{F}, which are appended to the parent molecular moiety through a nitrogen atom. R_{E} and R_{F} are each independently hydrogen, alkyl, and alkylcarbonyl. Representative examples of NR_{E}R_{F} include, but are not limited to, amino, methylamino, acetylamino, and acetylmethylamino.

The term “(NR_{E}R_{F})carbonyl” as used herein, means a NR_{E}R_{F} group, as defined herein, appended to the parent molecular moiety through a carbonyl group, as defined herein. Representative examples of (NR_{E}R_{F})carbonyl include, but are not limited to, aminocarbonyl, (methylamino)carbonyl, (dimethylamino)carbonyl, and (ethylmethylamino)carbonyl.

The term “oxo” as used herein, means a ═O moiety.

The terms “treat”, “treating” and “treatment” refer to a method of alleviating or abrogating a disease and/or its attendant symptoms.

By “pharmaceutically acceptable” it is meant the carrier, diluent or excipient must be compatible with the other ingredients of the formulation and not deleterious to the recipient thereof.

“Solvate” of a compound refers to a molecular complex of the solute (the compound) and the solvent.

The “subject” is defined herein to include animals such as mammals, including, but not limited to, primates (e.g., humans), cows, sheep, goats, horses, dogs, cats, rabbits, rats, mice and the like. In preferred embodiments, the subject is a human.

As used herein “chemotherapeutic agent” or “chemotherapy agent” or “antineoplastic agent” refer to an agent that reduces, prevents, and/or delays the growth of metastases or neoplasms, or kills neoplastic cells directly by necrosis or apoptosis in a pharmaceutically-effective amount, to reduce, prevent, and/or delay the growth of metastases or neoplasms in a subject with neoplastic disease.

“Chemotherapy” refers to treatments using chemotherapeutic agents, chemotherapy agents, or antineoplastic agents.

“Effective amount” or a “pharmaceutically-effective amount” in reference to the compounds or compositions of formula (I) or (II) refers to the amount sufficient to induce a desired biological, pharmacological, or therapeutic outcome in a subject.

“Chemotherapy-induced peripheral neuropathy” is a toxic neuropathy that results from the direct injury of the peripheral nervous system by a chemotherapeutic agent(s). CIPN can be acute or chronic. CIPN can be sensory, motor, autonomic, or a mixture of any of the three classes.

“Neurotoxic effects” and “neurotoxicity” refers to toxic substances altering the normal activity of the nervous system.

“Neuropathic pain” is the intractable pain caused by dysfunction in the peripheral or central nervous system.

The present invention provides a method of treating chemotherapy-induced peripheral neuropathy in a subject, comprising administering to the subject a therapeutically effective amount of a compound of formula (I) or (II) or a pharmaceutically acceptable salt or solvate thereof.

In another aspect, the present invention provides a method for prophylactic treatment of chemotherapy-induced peripheral neuropathy in a subject, comprising administering to the subject an effective amount of a compound of formula (I) or (II).

In another aspect, the present invention provides a method for mitigating neurotoxic effects of a chemotherapeutic agent, comprising administering to a subject an effective amount of a compound of formula (I) or (II).

In yet another aspect, the present invention provides a method for treating chemotherapy-induced neuropathic pain in a subject, comprising administering to the subject an effective amount of a compound of formula (I) or (II).

Compounds of formula (I) are inhibitors of poly(ADP-ribose)polymerase (PARP) and have been previously described in WO 2006-110816. Compounds of formula (II) similarly are PARP inhibitors and have been previously described in WO 2008/083027. Poly(ADP-ribose)polymerase has an essential role in facilitating DNA repair, controlling RNA transcription, mediating cell death, and regulating immune response. These actions make PARP inhibitors targets for a broad spectrum of disorders. (Virag L., et al., Pharmacol. Rev. 2002 54(3):375-429). In various preclinical cancer models and human clinical trials, PARP inhibitors have been shown to potentiate radiation and chemotherapy by increasing apoptosis of cancer cells, limiting tumor growth, decreasing metastasis, and prolonging the survival of tumor-bearing subjects. (WO 2007-084532; Donawho C. K., et al., Clin Cancer Res 2007 13(9):2728-37; Kummar S., et al., J Clin Oncol. 2009 27(16):2705-11).

In one embodiment, the present invention provides compounds of formula (I)

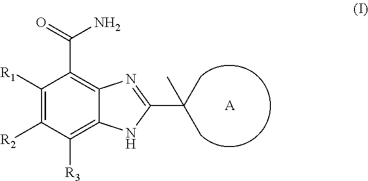

or a pharmaceutically acceptable salt or solvate thereof, wherein

R₁, R₂, and R₃ are independently selected from the group consisting of hydrogen, alkenyl, alkoxy, alkoxycarbonyl, alkyl, alkynyl, cyano, haloalkoxy, haloalkyl, halogen, hydroxy, hydroxyalkyl, nitro, NR_{A}R_{B}, and (NR_{A}R_{B})carbonyl;

A is a nonaromatic 4, 5, 6, 7, or 8-membered ring that contains 1 or 2 nitrogen atoms and, optionally, one sulfur or oxygen atom, wherein the nonaromatic ring is optionally substituted with 1, 2, or 3 substituents selected from the group consisting of alkenyl, alkoxy, alkoxyalkyl, alkoxycarbonyl, alkoxycarbonylalkyl, alkyl, alkynyl, aryl, arylalkyl, cycloalkyl, cycloalkylalkyl, cyano, haloalkoxy, haloalkyl, halogen, heterocycle, heterocyclealkyl, heteroaryl, heteroarylalkyl, hydroxy, hydroxyalkyl, nitro, NR_{C}R_{D}, (NR_{C}R_{D})alkyl, (NR_{C}R_{D})carbonyl, (NR_{C}R_{D})carbonylalkyl, (NR_{C}R_{D})sulfonyl, and oxo; and

R_{A}, R_{B}, R_{C}, and R_{D} are independently selected from the group consisting of hydrogen, alkyl, and alkycarbonyl.

In another embodiment of the invention, R₁, R₂, and R₃ are hydrogen or halogen; A is selected from the group consisting of

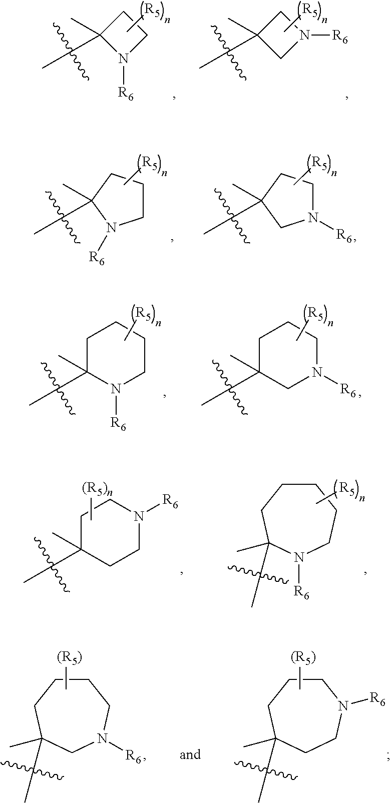

n is 0; R₆ is selected from the group consisting of hydrogen, alkenyl, alkoxyalkyl, alkoxycarbonyl, alkoxycarbonylalkyl, alkyl, alkynyl, aryl, arylalkyl, cycloalkyl, cycloalkylalkyl, heterocycle, heterocyclealkyl, heteroaryl, heteroarylalkyl, hydroxyalkyl, (NR_{C}R_{D})alkyl, (NR_{C}R_{D})carbonyl, (NR_{C}R_{D})carbonylalkyl, and (NR_{C}R_{D})sulfonyl; and R_{C} and R_{D} are independently selected from the group consisting of hydrogen and alkyl.

In another embodiment of the invention, A is selected from the group consisting of

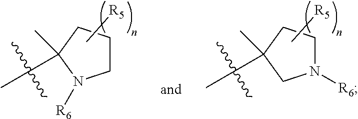

n is 0; R₁, R₂, and R₃ are hydrogen or halogen; R₆ is selected from the group consisting of hydrogen, alkyl, (NR_{C}R_{D})sulfonyl, arylalkyl, cycloalkyl, cycloalkylalkyl, heterocycle, and heteroarylalkyl, and R_{C} and R_{D} are independently selected from the group consisting of hydrogen and alkyl.

In yet another embodiment of the invention, A is selected from the group consisting of

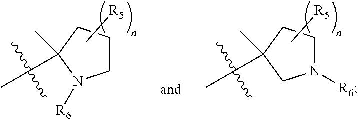

n is 0; R₁, R₂, and R₃ are hydrogen or halogen; and R⁶ is hydrogen.

In another embodiment of the invention, the compound of formula (I) is 2-(2-methylpyrrolidin-2-yl)-1H-benzimidazole-4-carboxamide. In yet another embodiment of the invention, the compound of formula (I) is 2-[(2R)-2-methylpyrrolidin-2-yl]-1H-benzimidazole-4-carboxamide. In yet another embodiment of the invention, the compound of formula (I) is 2-[(2S)-2-methylpyrrolidin-2-yl]-1H-benzimidazole-4-carboxamide.

In another embodiment of the invention, the compound of formula (I) is 6-fluoro-2-(2-methylpyrrolidin-2-yl)-1H-benzimidazole-4-carboxamide.

Another embodiment of the invention provides compounds of formula (II):

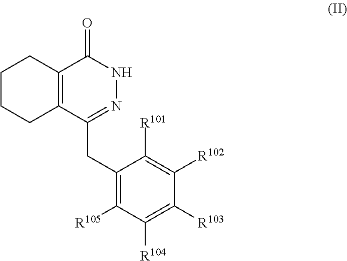

or a pharmaceutically acceptable salt or solvate thereof, wherein

R₁₀₁, R₁₀₄, and R₁₀₅ are H;

R₁₀₃ is F; and

R₁₀₂ is selected from pyrrolidinyl, oxazolyl, imidazolidinyl, isothiazolidinyl, piperidinyl, piperazinyl and azepanyl, wherein R₁₀₂ is substituted with one or two (O) substituents.

In another embodiment of the invention, the compound of formula (II) is 1-(2-fluoro-5-((4-oxo-3,4,5,6,7,8-hexahydrophthalazin-1-yl)methyl)phenyl)piperidine-2,6-dione. In yet another embodiment of the invention, the compound of formula (II) is 1-(2-fluoro-5-((4-oxo-3,4,5,6,7,8-hexahydrophthalazin-1-yl)methyl)phenyl)pyrrolidine-2,5-dione.

Compounds of formula (I) or (II) may contain asymmetrically substituted carbon atoms in the R or S configuration, wherein the terms “R” and “S” are as defined in Pure Appl. Chem. (1976) 45, 13-10. Compounds having asymmetrically substituted carbon atoms with equal amounts of R and S configurations are racemic at those atoms. Atoms having excess of one configuration over the other are assigned the configuration in excess, preferably an excess of about 85%-90%, more preferably an excess of about 95%-99%, and still more preferably an excess greater than about 99%. Accordingly, this invention is meant to embrace racemic mixtures and relative and absolute diastereoisomers of the compounds thereof.

This invention also is directed, in part, to all salts of the compounds of formula (I) or (II) and methods of their use. A salt of a compound may be advantageous due to one or more of the salt's properties, such as, for example, enhanced pharmaceutical stability in differing temperatures and humidities, or a desirable solubility in water or other solvents. Where a salt is intended to be administered to a patient (as opposed to, for example, being in use in an in vitro context), the salt preferably is pharmaceutically acceptable and/or physiologically compatible. The term “pharmaceutically acceptable” is used adjectivally in this patent application to mean that the modified noun is appropriate for use as a pharmaceutical product or as a part of a pharmaceutical product. Pharmaceutically acceptable salts include salts commonly used to form alkali metal salts and to form addition salts of free acids or free bases. In general, these salts typically may be prepared by conventional means by reacting, for example, the appropriate acid or base with a compound of the invention.

Pharmaceutically acceptable acid addition salts of the compounds of formula (I) or (II) can be prepared from an inorganic or organic acid. Examples of often suitable inorganic acids include hydrochloric, hydrobromic, hydroiodic, nitric, carbonic, sulfuric, and phosphoric acid. Suitable organic acids generally include, for example, aliphatic, cycloaliphatic, aromatic, araliphatic, heterocyclic, carboxylic, and sulfonic classes of organic acids. Specific examples of often suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), ethanesulfonate, benzenesulfonate, pantothenate, 2-hydroxyethanesulfonate, sulfanilate, cyclohexylaminosulfonate, algenic acid, beta-hydroxybutyric acid, galactarate, galacturonate, adipate, alginate, bisulfate, butyrate, camphorate, camphorsulfonate, cyclopentanepropionate, dodecylsulfate, glycoheptanoate, glycerophosphate, heptanoate, hexanoate, nicotinate, oxalate, palmoate, pectinate, 2-naphthalesulfonate, 3-phenylpropionate, picrate, pivalate, thiocyanate, tosylate, and undecanoate.

Pharmaceutically acceptable base addition salts of the compounds of formula (I) or (II) include, for example, metallic salts and organic salts. Preferred metallic salts include alkali metal (group Ia) salts, alkaline earth metal (group IIa) salts, and other physiologically acceptable metal salts. Such salts may be made from aluminum, calcium, lithium, magnesium, potassium, sodium, and zinc. Preferred organic salts can be made from amines, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups can be quaternized with agents such as lower alkyl (C₁-C₆) halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl, and stearyl chlorides, bromides, and iodides), arylalkyl halides (e.g., benzyl and phenethyl bromides), and others.

This invention also is directed, in part, to all compositions of the compounds of formula (I) or (II) and methods of their use. Compounds having formula (I) or (II) may be administered with or without an excipient. Excipients include, but are not limited to, encapsulators and additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, mixtures thereof and the like.

Excipients for preparation of compositions comprising a compound having formula (I) to be administered orally include, but are not limited to, agar, alginic acid, aluminum hydroxide, benzyl alcohol, benzyl benzoate, 1,3-butylene glycol, carbomers, castor oil, cellulose, cellulose acetate, colloidal silica, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl celluose, isopropanol, isotonic saline, lactose, magnesium hydroxide, magnesium stearate, malt, mannitol, microcrystalline cellulose, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, titanium dioxide, tragacanth, tetrahydrofurfuryl alcohol, triglycerides, water, mixtures thereof and the like.

Total daily dose of the compositions of the invention to be administered to a human or other mammal host in single or divided doses may be in amounts, for example, from 0.0001 to 300 mg/kg body weight daily and more usually 1 to 300 mg/kg body weight. The dose, from 0.0001 to 300 mg/kg body, may be given twice a day.

In one embodiment of the invention, the dose of compound of formula (I) or (II), or a pharmaceutically acceptable salt or solvate thereof, is in the range of 5 to 400 mg, the range of 10 to 200 mg, the range of 10 to 100 mg, or the range of 10 to 50 mg. In a further embodiment of the invention, the dose of a compound of formula (I) or (II), or a pharmaceutically acceptable salt or solvate thereof, is about 5 mg, 10 mg, 20 mg, 40 mg, 50 mg, 60 mg, 80 mg, or 100 mg. The dose can be administered once a day or twice a day. Alternatively, the dose can be administered twice a week. Alternatively, the dose can be administered once a week.

In one embodiment, the chemotherapy-induced peripheral neuropathy is sensory. In one embodiment, the neuropathy presents as distal axonopathy. In another embodiment, the neuropathy presents as dysesthesia, paraesthesia, burning, numbness, and/or pain.

In one embodiment, the chemotherapy-induced peripheral is motor. In another embodiment, the neuropathy presents as myoatrophy. In another embodiment, the neuropathy presents with loss of distal deep tendon reflexes.

In one embodiment, chemotherapy-induced peripheral neuropathy is autonomic.

In one embodiment, the subject has an elevated risk of developing chemotherapy-induced peripheral neuropathy. Subjects with an elevated risk of developing CIPN have preexisting conditions including diabetes, nutritional deficiency, alcoholism, and previous exposure to neurotoxic chemotherapy. In another embodiment, the subject has a past history of neuropathy. The previous neuropathy may have been caused by diabetes, nutritional deficiency, alcoholism, hereditary disease and/or neurotoxic chemotherapy.

In one embodiment, the present invention further comprises the step of administering one or more chemotherapeutic agents.

Chemotherapeutic agent or agents may include, for example, antimetabolites (i.e., folate antagonists, purine antagonists, and pyrimidine antagonists), bleomycins, DNA alkylating agents (i.e., nitrosoureas, cross linking agents, and alkyating agents), hormones, aromatase inhibitors, monoclonal antibodies, antibiotics, platinum complexes, protesome inhibitors, taxane analogs, vinca alkaloids, topoisomerase inhibitors (i.e., anthracyclines, camptothecins, podophyllotoxins), tyrosine kinase inhibitors, or a combination thereof.

In another embodiment, chemotherapeutic agents may include, for example, a platinum complex, a vinca analog, a taxane analog, an alkylating agent, an antimetabolite, a proteasome inhibitor, or a combination thereof.

Platinum complexes may include, for example, cisplatin, oxaliplatin, eptaplatin, lobaplatin, nedaplatin, carboplatin, satraplatin, picoplatin and the like.

Vinca alkaloids may include, for example, vincristine, vinblastine, vinorelbine, vindesine, and the like.

Taxanes may include, for example, paclitaxel, docetaxel, and various formulations and analogs thereof.

Alkylating agents may include, for example, dacarbazine, procarbazine, temozolamide, thiotepa, mechlorethamine, chlorambucil, L-phenylalanine mustard, melphalan, ifosphamide, cyclophosphamide, mefosphamide, perfosfamide, trophosphamide, busulfan, carmustine, lomustine, thiotepa, semustine, and the like.

Antimetabolites include pemetrexed disodium, 5 azacitidine, capecitabine, carmofur, cladribine, clofarabine, cytarabine, cytarabine ocfosfate, cytosine arabinoside, decitabine, deferoxamine, doxifluridine, eflornithine, enocitabine, ethnylcytidine, fludarabine, 5 fluorouracil alone or in combination with leucovorin, gemcitabine, hydroxyurea, melphalan, mercaptopurine, 6 mercaptopurine riboside, methotrexate, mycophenolic acid, nelarabine, nolatrexed, ocfosfate, pelitrexol, pentostatin, raltitrexed, Ribavirin, triapine, trimetrexate, S-1, tiazofurin, tegafur, TS-1, vidarabine, UFT and the like.

Proteasome inhibitors may include, for example, bortezomib.

Topoisomerase inhibitors include aclarubicin, 9-aminocamptothecin, amonafide, amsacrine, becatecarin, belotecan, irinotecan hydrochloride, camptothecin, dexrazoxine, diflomotecan, edotecarin, epirubicin, etoposide, exatecan, 10-hydroxycamptothecin, gimatecan, lurtotecan, mitoxantrone, orathecin, pirarbucin, pixantrone, rubitecan, sobuzoxane, SN-38, tafluposide, topotecan and the like.

In another embodiment, chemotherapeutic agents are bortezomib, carboplatin, cisplatin, gemcitabine, misonidazole, oxaliplatin, procarbazine, thalidomide, docetaxel, hexamethylmelamine, paclitaxel, vincristine, vinblastine, or vinorelbine.

In one embodiment of the invention, the chemotherapeutic agent is carboplatin and the compound of formula (I) is 2-[(2R)-2-methylpyrrolidin-2-yl]-1H-benzimidazole-4-carboxamide. Yet another embodiment further comprises the chemotherapeutic agent topotecan. Yet another embodiment further comprises the chemotherapeutic agent cyclophosphamide.

In one embodiment of the invention, the chemotherapeutic agent is cisplatin and the compound of formula (I) is 2-[(2R)-2-methylpyrrolidin-2-yl]-1H-benzimidazole-4-carboxamide. Yet another embodiment further comprises the chemotherapeutic agent cyclophosphamide.

In one embodiment of the invention, the chemotherapeutic agent is oxaliplatin and the compound of formula (I) is 2-[(2R)-2-methylpyrrolidin-2-yl]-1H-benzimidazole-4-carboxamide. Yet another embodiment further comprises the chemotherapeutic agent capecitabine. Yet another embodiment further comprises the chemotherapeutic agents 5-fluorouracil and leucovorin.

In one embodiment of the invention, the chemotherapeutic agent is paclitaxel and the compound of formula (I) is 2-[(2R)-2-methylpyrrolidin-2-yl]-1H-benzimidazole-4-carboxamide. Yet another embodiment further comprises the chemotherapeutic agent cisplatin. Yet another embodiment further comprises the chemotherapeutic agents doxorubicin and cyclophosphamide.

In one embodiment of the invention, the chemotherapeutic agent is docetaxel and the compound of formula (I) is 2-[(2R)-2-methylpyrrolidin-2-yl]-1H-benzimidazole-4-carboxamide. Yet another embodiment further comprises the chemotherapeutic agents doxorubicin and cyclophosphamide. Yet another embodiment further comprises the chemotherapeutic agents cisplatin and fluorouracil.

In one embodiment of the invention, the chemotherapeutic agent is vinorelbine and the compound of formula (I) is 2-[(2R)-2-methylpyrrolidin-2-yl]-1H-benzimidazole-4-carboxamide. Yet another embodiment further comprises the chemotherapeutic agent cisplatin.

In one embodiment of the invention, the chemotherapeutic agents are carboplatin and docetaxel and the compound of formula (I) is 2-[(2R)-2-methylpyrrolidin-2-yl]-1H-benzimidazole-4-carboxamide.

In one embodiment of the invention, the chemotherapeutic agents are cisplatin and docetaxel and the compound of formula (I) is 2-[(2R)-2-methylpyrrolidin-2-yl]-1H-benzimidazole-4-carboxamide.

In one embodiment of the invention, the chemotherapeutic agents are carboplatin and paclitaxel and the compound of formula (I) is 2-[(2R)-2-methylpyrrolidin-2-yl]-1H-benzimidazole-4-carboxamide. Yet another embodiment further comprises the chemotherapeutic agent bevacizumab.

In one embodiment of the invention, the chemotherapeutic agents are cisplatin and paclitaxel and the compound of formula (I) is 2-[(2R)-2-methylpyrrolidin-2-yl]-1H-benzimidazole-4-carboxamide.

In one embodiment of the invention, the chemotherapeutic agents are carboplatin and gemcitabine and the compound of formula (I) is 2-[(2R)-2-methylpyrrolidin-2-yl]-1H-benzimidazole-4-carboxamide.

In one embodiment of the invention, the chemotherapeutic agents are cisplatin and gemcitabine and the compound of formula (I) is 2-[(2R)-2-methylpyrrolidin-2-yl]-1H-benzimidazole-4-carboxamide.

In one embodiment of the invention, the chemotherapeutic agents are cisplatin and vinorelbine and the compound of formula (I) is 2-[(2R)-2-methylpyrrolidin-2-yl]-1H-benzimidazole-4-carboxamide.

In another embodiment, the chemotherapeutic agent or agents is administered for the treatment of cancer.

In one embodiment of the invention, the cancer being treated is acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute t-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myleogeneous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenström's macroglobulinemia, testicular tumors, uterine cancer and Wilms' tumor.

In yet another embodiment of the invention, the cancer being treated is selected from the group consisting of ovarian cancer, cervical cancer, colorectal cancer, prostate cancer, breast cancer, gastric adenocarcinoma, head and neck cancer, testicular cancer, leukemia, neuroblastoma, Hodgkin's lymphoma, non-Hodgkin's lymphoma, and non-small cell lung cancer.

The administration of a compound of formula (I) or (II) or a pharmaceutically acceptable salt or solvate thereof, and compositions and formulations thereof, may be prior to, immediately prior to, during, immediately subsequent to or subsequent to the administration of the one or more chemotherapeutic agents. The compound of formula (I) or (II) can be administered prophylactically before CIPN is established or for treating established CIPN. The established CIPN can be acute or chronic.

Cisplatin can be administered at a range of 20 mg/m² to 140 mg/m² in cycles of 1, 2, 3, 4, 5, 6, 7, or 8. For example, cisplatin can be administered at 20 mg/m² daily for five days per cycle. Cisplatin can be administered at 75 to 100 mg/m² once per cycle every four weeks (Day 1). Cisplatin can be administered 50 to 70 mg/m² once per cycle every three to four weeks (Day 1).

Carboplatin can be administered at about 300 mg/m² or less or at about 360 mg/m² or less once per cycle every three to four weeks (Day 1). Carboplatin can be administered in cycles of 1, 2, 3, 4, 5, 6, 7, or 8.

Oxaliplatin can be administered at about 85 mg/m² or less one per cycle every 2 weeks. Oxaliplatin can be administered in cycles of 1, 2, 3, 4, 5, 6, 7, or 8.

Docetaxel can be administered at about 60 mg/m² to about 100 mg/m² in cycles of 1, 2, 3, 4, 5, 6, 7, or 8. For example, docetaxel can be administered at 75 mg/m² once per cycle every three weeks (Day 1).

Paclitaxel can be administered at a range of about 100 mg/m² to about 175 mg/m² in cycles of 1, 2, 3, 4, 5, 6, 7, or 8. Paclitaxel can be administered at about 100 mg/m² once per cycle every 3 weeks (Day 1). Paclitaxel can be administered at about 135 mg/m² once per cycle every 3 weeks (Day 1). Paclitaxel can be administered at about 175 mg/m² once per cycle every 3 weeks (Day 1).

Vincristine can be administered at a range of about 0.4 mg/m² to 1.4 mg/m² once per cycle every one to four weeks (Day 1). Vincrinstine can be administered in cycles of 1, 2, 3, 4, 5, 6, 7, or 8.

Vinblastine can be administered at a range of about 3.7 mg/m² to about 18.5 mg/m² once per cycle every one to four weeks (Day 1). For example, vinblastine can be administered at 3.7 mg/m², 5.5 mg/m2, 7.4 mg/m², 9.25 mg/m², or 11.1 mg/m². Vinblastine can be administered in cycles of 1, 2, 3, 4, 5, 6, 7, or 8.

Vinorelbine can be administered at a range of about 25 m g/m² to about 120 mg/m² once per cycle every one to six weeks (Day 1). For example, vinorelbine can be administered at 30 mg/m². Vinorelbine can be administered in cycles of 1, 2, 3, 4, 5, 6, 7, or 8.

In one embodiment, compounds of formula (I) or (II) and compositions and formulations thereof are administered once a day during the treatment cycle wherein a chemotherapeutic agent or agent(s) is administered at Day 1 of the cycle, wherein a cycle is 5 days, 1 week, 2 weeks, 3 weeks, 4 weeks, 5 weeks or 6 weeks.

In one embodiment, compounds of formula (I) or (II) and compositions and formulations thereof are administered twice a day during the treatment cycle wherein a chemotherapeutic agent or agent(s) is administered at Day 1 of the cycle, wherein a cycle is 5 days, 1 week, 2 weeks, 3 weeks, 4 weeks, 5 weeks or 6 weeks.

In one embodiment, compounds of formula (I) or (II) and compositions and formulations thereof are administered twice a week during the treatment cycle wherein a chemotherapeutic agent or agent(s) is administered at Day 1 of the cycle, wherein a cycle is 5 days, 1 week, 2 weeks, 3 weeks, 4 weeks, 5 weeks or 6 weeks.

In one embodiment, compounds of formula (I) or (II) and compositions and formulations thereof are administered once a week during the treatment cycle wherein a chemotherapeutic agent or agent(s) is administered at Day 1 of the cycle, wherein a cycle is 5 days, 1 week, 2 weeks, 3 weeks, 4 weeks, 5 weeks or 6 weeks.

In one embodiment, compounds of formula (I) or (II) and compositions and formulations thereof are administered once a week during the treatment cycle wherein a chemotherapeutic agent or agent(s) is administered at Day 1 of the cycle, wherein a cycle is 5 days, 1 week, 2 weeks, 3 weeks, 4 weeks, 5 weeks or 6 weeks.

In another embodiment, compounds of formula (I) or (II) and compositions and formulations thereof are administered at least one day prior to chemotherapy. In another embodiment, compounds of formula (I) or (II) and compositions and formulations thereof are administered for two days prior to chemotherapy. In another embodiment, compounds of formula (I) and compositions and formulations thereof are administered for one week prior to chemotherapy. In yet another embodiment, compounds of formula (I) or (II) and compositions and formulations thereof are administered immediately prior to each chemotherapy treatment. In yet another embodiment, compounds of formula (I) or (II) and compositions and formulations thereof are administered simultaneously with each chemotherapy treatment. In yet another embodiment, compounds of formula (I) or (II) and compositions and formulations thereof are administered subsequent to chemotherapy.

The invention further allows for administration of higher dose of chemotherapy. Additionally, the invention allows for administration of additional cycles of chemotherapy. The invention also allows for reduction of time between cycles of chemotherapy.

The severity of the incidence of CIPN is reflected in the grade, i.e., 0, 1, 2, 3, or 4. The scale escalates from grade 0, normal and asymptomatic, to grade 4, disabling and/or life-threatening. (Postma T. J., Annals of Oncology 1998 9:739-744). Grade 3 requires corrective measures, including dose reduction and/or delays.

There are multiple Common Toxicity Criteria (CTC) scales used in clinical practice to evaluate the severity of CIPN: World Health Organization (WHO) scale, Eastern Cooperative Oncology Group (ECOG) scale, National Cancer Institute—Common Toxicity Criteria (NCI-CTC), and Ajani scale. (Cavaletti G., et al., European Journal of Cancer 2010 46:479-494). The scales represent a combination of objective assessment and the patients' perception of CIPN effects.

One embodiment of the invention provides methods of treating, including treating prophylactically, chemotherapy-induced peripheral neuropathy with a compound of formula (I), wherein the incidence of grade 3 or 4 CIPN is decreased. In another embodiment, the incidence of grade 1 or 2 CIPN is decreased. In another embodiment, the incidence of grade 3 or 4 CIPN is decreased to grade 1 or 2 CIPN. In another embodiment, the incidence of grade 2 CIPN is decreased to grade 1.

The present invention further provides a method for mitigating neurotoxic effects of a chemotherapeutic agent, wherein incidence of grade 3 or 4 CIPN is decreased. In another embodiment, the incidence of grade 1 or 2 CIPN is decreased. In another embodiment, the incidence of grade 3 or 4 CIPN is decreased to grade 1 or 2 CIPN. In another embodiment, the incidence of grade 2 CIPN is decreased to grade 1.

Alternatively, CIPN can be evaluated with a quality of life assessment. One such assessment is the European Organization of Research and Treatment of Cancer (EORTC) QLQ-CIPN20 questionnaire. (Cavaletti G., et al., European Journal of Cancer 2010 46:479-494).

In one embodiment of the invention, CIPN is improved on EORTC QLQ-CIPN 20 questionnaire.

One embodiment of the invention provides methods of treating chemotherapy-induced neuropathic pain with a compound of formula (I) or (II). Neuropathic pain is the intractable pain caused by dysfunction in the peripheral or central nervous system.

Pain can be evaluated with a quality of life assessment. One such assessment is the European Organization of Research and Treatment of Cancer (EORTC) EORTC QLQ-C30/L13 questionnaire.

In one embodiment of the invention, the pain is decreased based on the assessment of the EORTC QLQ-C30/L13 questionnaire.

In one embodiment of the invention, the pain is peripheral neuropathic pain or central neuropathic pain.

In another embodiment of the invention, the pain is chronic or acute.

In another embodiment of the invention, the use of supportive care for pain is reduced. Supportive care includes, for example, NSAIDS or opioids.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1

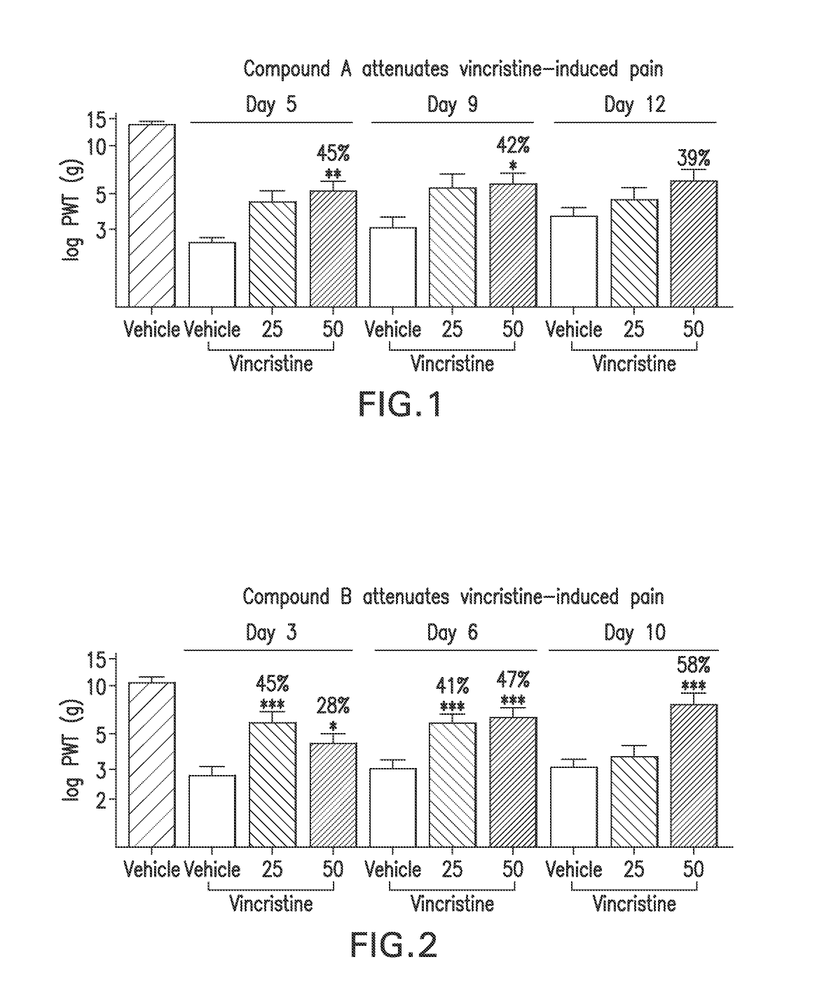

Rats were administered a PARP Inhibitor, 2-[(2R)-2-methylpyrrolidin-2-yl]-1H-benzimidazole-4-carboxamide (Compound A) or 6-fluoro-2-(2-methylpyrrolidin-2-yl)-1H-benzimidazole-4-carboxamide (Compound B), at doses of 25 mg/kg/day or 50 mg/kg/day (i.p.), for two days prior to the initiation of vincristine. After two days or pre-dosing with the PARP inhibitor, two minipumps were implanted in rats. Vincristine was administered via a subcutaneous mini-osmotic pump that delivered 30 ug/kg/day (i.v) for twelve days. PARP Inhibitor, Compound A or Compound B, or vehicle was administered via a subcutaneous mini-osmotic pump that delivered 25 mg/kg/day, 50 mg/kg/day, or vehicle (i.p.) for twelve days. A positive control group of rats receiving vincristine were dosed acutely with morphine (6 mg/kg, i.p.) on each day of testing. A negative control group of rats received saline. Mechanical threshold was determined for all rats using von Frey monofilaments at 5, 9 and 12 days following initiation of vincristine administration (days 7, 11 and 14 of compound delivery, respectively) for Compound A, and at 3, 6 and 10 days following initiation of vincristine administration (days 5, 8 and 12 of compound delivery, respectively) for Compound B. Mechanical allodynia was observed on all testing days in rats treated with vincristine compared to the naïve group. Morphine fully reversed mechanical allodynia on all testing days. PARP inhibitors, Compound A or Compound B, attenuated development of mechanical allodynia in the vincristine model of chemotherapy-induced pain (FIG. 1 and FIG. 2).

### Example 2

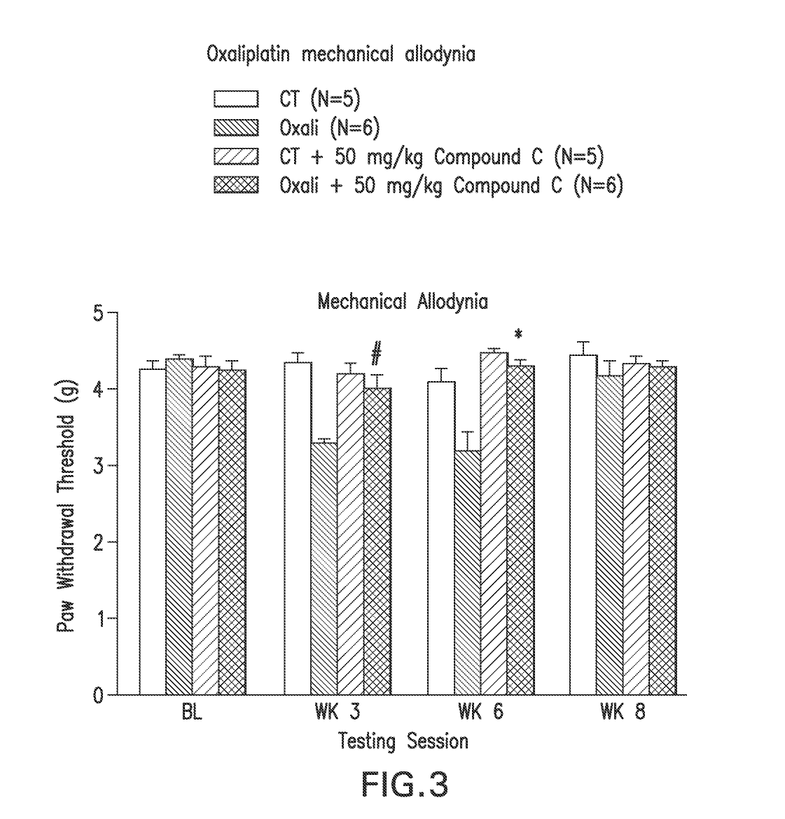

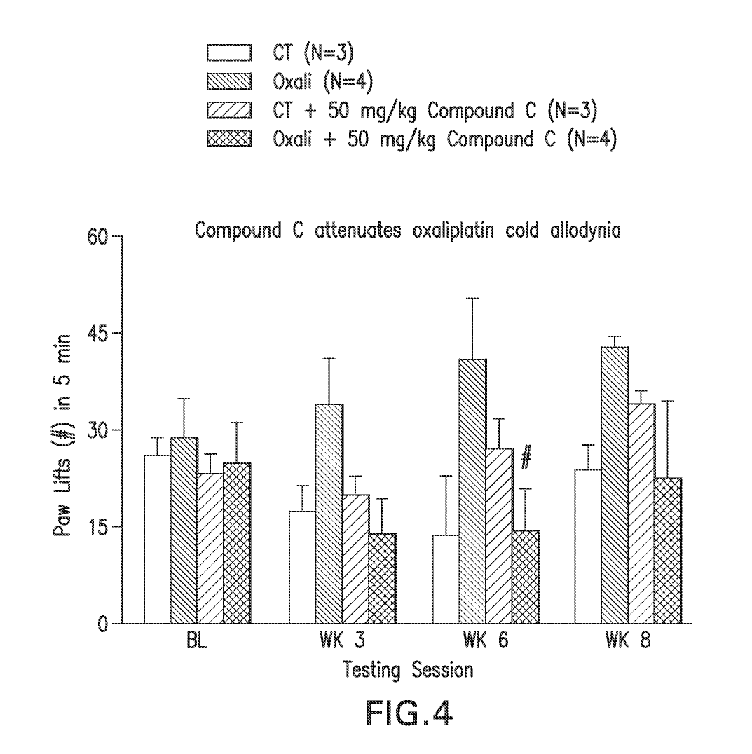

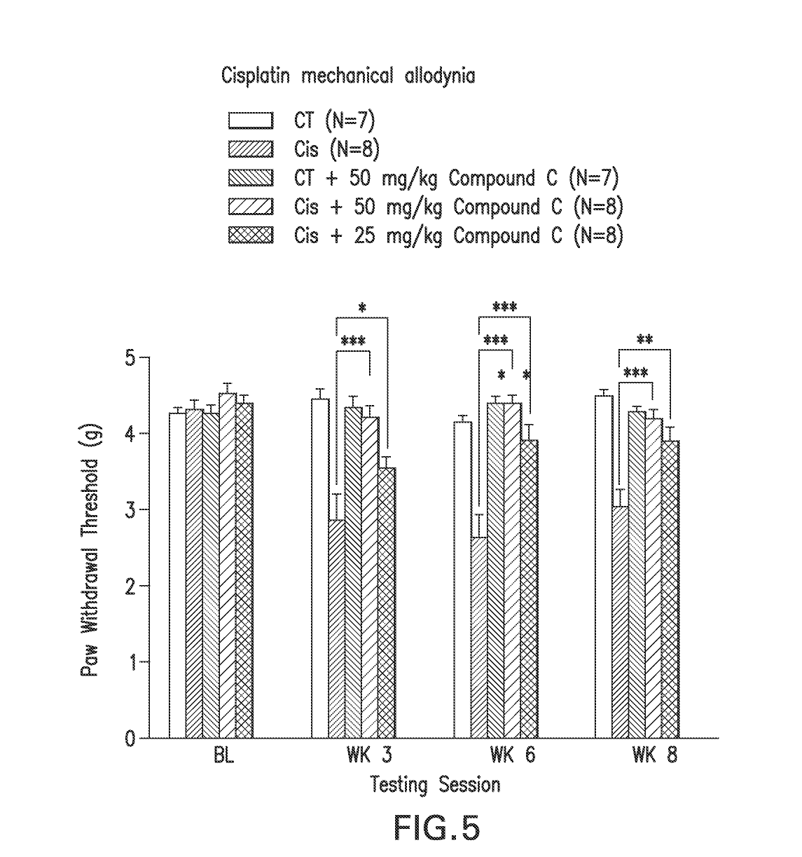

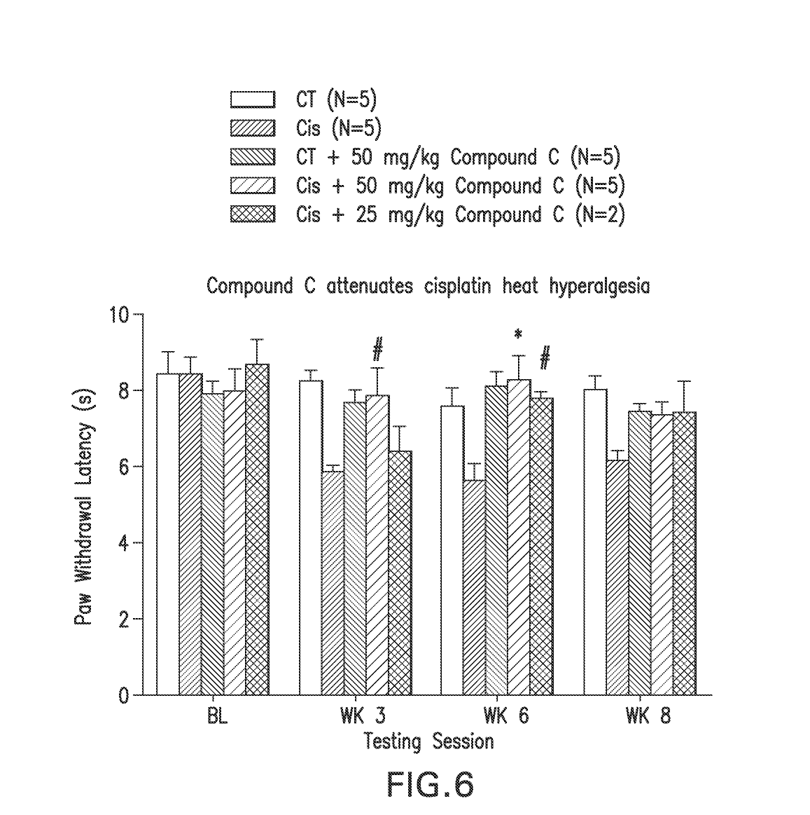

Mice were administered a PARP Inhibitor, 2-[(2S)-2-methylpyrrolidin-2-yl]-1H-benzimidazole-4-carboxamide (Compound C) at doses of 25 mg/kg/day or 50 mg/kg/day (i.p.) for two days prior to the initiation of cisplatin. The 50 mg/kg dose of Compound C was administered (i.p.) for two days prior to oxaliplatin administration. After two days of pre-dosing with the PARP Inhibitor, mice were co-administered Compound C with cisplatin or oxaliplatin for 5 days (daily injections, i.p.), followed by 5 days off, followed by 5 daily injections (i.p.). Cumulative dose of cisplatin was 23 mg/kg. Cumulative dose of oxaliplatin was 30 mg/kg. Behavioral assays were performed on all groups of mice before dosing, and then at weeks 3, 6, and 8. Behavioral assays including determining mechanical threshold with von Frey monofilaments, determining, latency to paw withdrawal from a radiant heat source, and number of paw lifts from a cold plate. Compound C attenuated development of mechanical allodynia in the cisplatin model at weeks 3, 6, and 8, and in the oxaliplatin model at weeks 3 and 6 (FIG. 3 and FIG. 4). Compound C attenuated development of heat hyperalgesia in the cisplatin model at weeks 3 and 6. Compound C attenuated development of cold hyperalgesia in the oxaliplatin model at week 6 (FIG. 5 and FIG. 6).

### Example 3

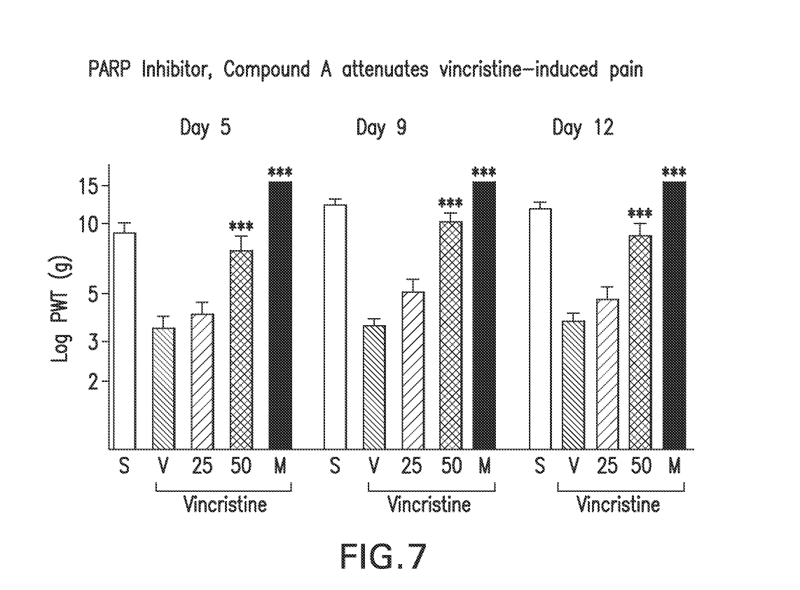

Rats were administered a PARP Inhibitor, 2-[(2R)-2-methylpyrrolidin-2-yl]-1H-benzimidazole-4-carboxamide (Compound A), at doses of 25 mg/kg/day or 50 mg/kg/day (i.p., bid) for two days prior to the initiation of vincristine. After two days or pre-dosing with ABT-888, two minipumps were implanted in rats. Vincristine was administered via a subcutaneous mini-osmotic pump that delivered 30 ug/kg/day (i.v) for twelve days. Compound A or vehicle was administered via a subcutaneous mini-osmotic pump that delivered 25 mg/kg/day or 50 mg/kg/day (i.p.) for twelve days. A positive control group of rats receiving vincristine were dosed with acutely morphine (6 mg/kg, i.p.) on each day of testing. A negative control group of rats received saline. Mechanical threshold was determined for all rats using von Frey monofilaments on 5, 9 and 12 days following initiation of vincristine administration (days 7, 11 and 14 of compound delivery, respectively). Mechanical allodynia was observed on all testing days in rats treated with vincristine compared to the naïve group. Morphine fully reversed mechanical allodynia on all testing days. Compound A attenuated development of mechanical allodynia in the vincristine model of chemotherapy-induced pain (FIG. 7).

### Example 4

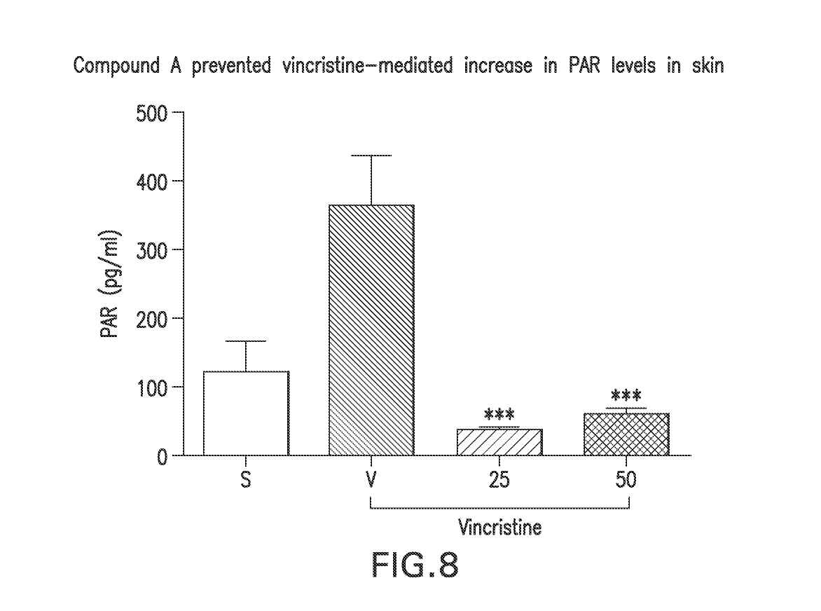

Following behavioral testing on day 12 in Example 3, skin biopsies were taken from glabrous hindpaw skin from rats in the naïve, vincristine, and vincristine+Compound A groups (n=5 per group). PAR levels were assessed using ab ELISA assay to measure pADPr as previously described (Liu et al., 2008). PAR levels in skin were increased by vincristine, as compared to the saline group. Pretreatment with Compound A (25 mg/kg and 50 mg/kg) significantly reduced vincristine-mediated PAR activation in rat glabrous skin (FIG. 8).

### Example 5

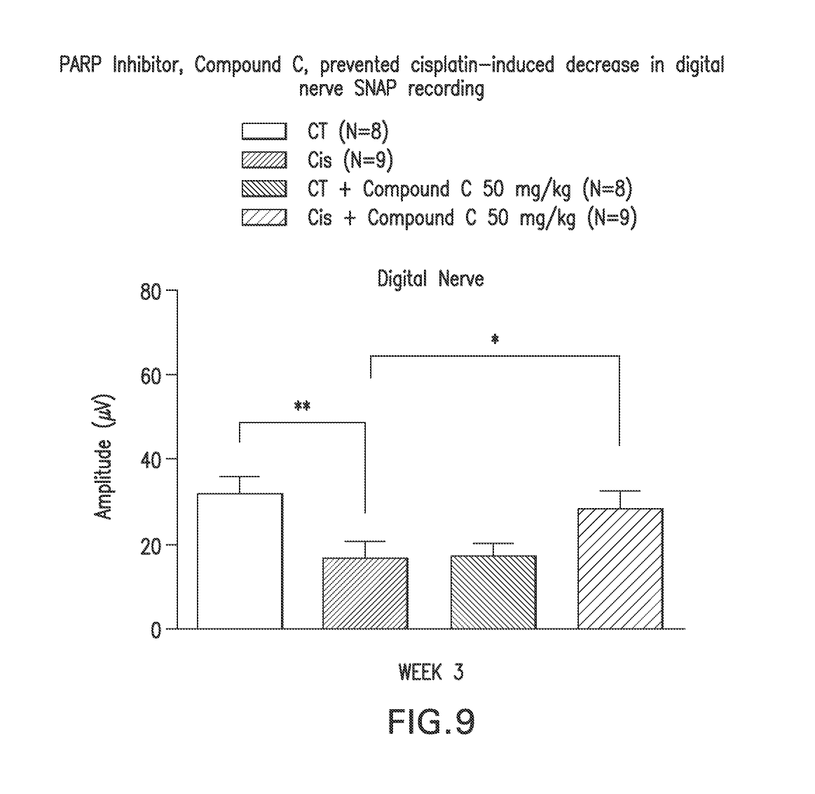

Mice were administered a PARP Inhibitor, 2-[(2S)-2-methylpyrrolidin-2-yl]-1H-benzimidazole-4-carboxamide (Compound C), at doses of 25 mg/kg/day or 50 mg/kg/day (i.p., bid), for two days prior to the initiation of cisplatin. After two days or pre-dosing with Compound C, mice were co-dosed with the PARP inhibitor (25 mg/kg/day or 50 mg/kg/day) and cisplatin (2.3 mg/kg/day, i.p.). The dosing regimen consisted for 5 daily co-injections, followed by 5 days off, and then a repeat of 5 daily co-injection. Nerve conduction studies were performed after the final injections in week 3. Sensory nerve action potential (SNAP) recordings were made from the digital nerve. Cisplatin induced a decrease in amplitude in SNAP recording from the digital nerve, an effect that was prevented by 2-[(2S)-2-methylpyrrolidin-2-yl]-1H-benzimidazole-4-carboxamide (Compound C) treatment (FIG. 9).

### Example 6

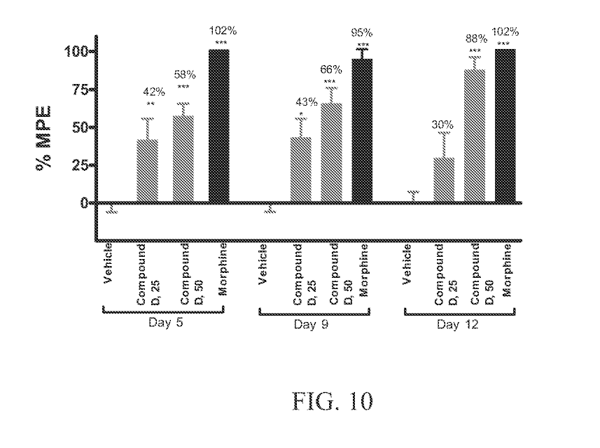

Vincristine (i.v, 30 mg kg/day) was administered by mini-pump for twelve days. Rats were divided into four groups: Group 1: Vehicle; Group 2: 1-(2-fluoro-5-((4-oxo-3,4,5,6,7,8-hexahydrophthalazin-1-yl)methyl)phenyl)pyrrolidine-2,5-dione (Compound D), 25 mg/kg/day; Group 3: Compound D, 50 mg/kg/day; Group 4: Vehicle+Positive control (morphine). Groups 2 and 3 were pretreated with their respective dose of Compound D (either 25 mg/kg or 50 mg/kg, ip) for two days prior to co-dosing with vincristine. Rats in the positive control group were dosed with vehicle by mini-pump and were acutely administered morphine on day of testing. Mechanical threshold was determined using von Frey monofilaments on days 5, 9, and 12 post-mini-pump implantation. Plasma and brain were collected on day 12. Compound D attenuated vincristine-induced mechanical allodynia. Anti-nociceptive efficacy was dose-dependent and observed on days 5, 9, and 12 (FIG. 10).

What is claimed is:

1. A method for treating chemotherapy-induced peripheral neuropathy in a subject, comprising administering to the subject a therapeutically effective amount of a compound of Formula (I) 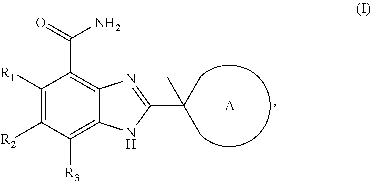
or a pharmaceutically acceptable salt or solvate thereof, wherein
R₁, R₂, and R₃ are independently selected from the group consisting of hydrogen, alkenyl, alkoxy, alkoxycarbonyl, alkyl, alkynyl, cyano, haloalkoxy, haloalkyl, halogen, hydroxy, hydroxyalkyl, nitro, NR_{A}R_{B}, and (NR_{A}R_{B})carbonyl;
A is a nonaromatic 4, 5, 6, 7, or 8-membered ring that contains 1 or 2 nitrogen atoms and, optionally, one sulfur or oxygen atom, wherein the nonaromatic ring is optionally substituted with 1, 2, or 3 substituents selected from the group consisting of alkenyl, alkoxy, alkoxyalkyl, alkoxycarbonyl, alkoxycarbonylalkyl, alkyl, alkynyl, aryl, arylalkyl, cycloalkyl, cycloalkylalkyl, cyano, haloalkoxy, haloalkyl, halogen, heterocycle, heterocyclealkyl, heteroaryl, heteroarylalkyl, hydroxy, hydroxyalkyl, nitro, NR_{C}R_{D}, (NR_{C}R_{D})alkyl, (NR_{C}R_{D})carbonyl, (NR_{C}R_{D})carbonylalkyl, (NR_{C}R_{D})sulfonyl, and oxo; and
R_{A}, R_{B}, R_{C}, and R_{D} are independently selected from the group consisting of hydrogen, alkyl, and alkycarbonyl.

2. The method of claim 1, wherein the treatment is prophylactic treatment.

3. The method of claim 1, wherein A is selected from the group consisting of

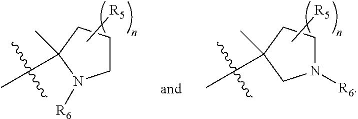

4. The method of claim 1, wherein R¹, R², and R³ are independently hydrogen or halogen;
R⁶ is hydrogen; and
n is 0.

5. The method of claim 1, wherein the compound of formula (I) is 2-(2-methylpyrrolidin-2-yl)-1H-benzimidazole-4-carboxamide.

6. The method of claim 1, wherein the compound of formula (I) is 2-[(2R)-2-methylpyrrolidin-2-yl]-1H-benzimidazole-4-carboxamide.

7. The method of claim 1, wherein the compound of formula (I) is 2-[(2S)-2-methylpyrrolidin-2-yl]-1H-benzimidazole-4-carboxamide.

8. The method of claim 1, further comprising administering one or more chemotherapeutic agents.

9. The method of claim 8, wherein the chemotherapeutic agent or agents is utilized for the treatment of cancer.

10. The method of claim 8, wherein the chemotherapeutic agent is selected from the group consisting of bortezomib, carboplatin, cisplatin, gemcitabine, misonidazole, oxaliplatin, procarbazine, thalidomide, docetaxel, hexamethylmelamine, paclitaxel, vincristine, vinblastine, or vinorelbine.

11. The method of claim 10, wherein the chemotherapeutic agent is carboplatin.

12. The method of claim 10, wherein the chemotherapeutic agent is cisplatin.

13. The method of claim 10, wherein the chemotherapeutic agent is paclitaxel.

14. The method of claim 10, wherein the chemotherapeutic agent is vinorelbine.

15. The method of claim 10, wherein the chemotherapeutic agents are cisplatin and docetaxel.

16. The method of claim 10, wherein the chemotherapeutic agents are carboplatin and docetaxel.

17. The method of claim 10, wherein the chemotherapeutic agents are cisplatin and gemcitabine.

18. The method of claim 10, wherein the chemotherapeutic agents are carboplatin and gemcitabine.

19. The method of claim 9, wherein the cancer is selected from the group consisting of ovarian cancer, cervical cancer, colorectal cancer, prostate cancer, breast cancer, testicular cancer, leukemia, neuroblastoma, Hodgkin's lymphoma, non-Hodgkin's lymphoma, and non-small cell lung cancer.

20. The method of claim 9, wherein the cancer is selected from the group consisting of ovarian cancer, breast cancer, and non-small cell lung cancer.

21. The method of claim 8, wherein the compound of formula (I) is administered before the administration of the chemotherapeutic agent.

22. The method of claim 8, wherein the compound of formula (I) is administered during the administration of the chemotherapeutic agent.

23. The method of claim 8, wherein the compound of formula (I) is administered after the administration of the chemotherapeutic agent.
